(19) United States
(12) Patent Application Publication
(10) Pub. No.: US 2006/0183796 A1
(43) Pub. Date: Aug. 17, 2006

(54) Method for the medicinal prophylaxis of cholinesterase inhibitor intoxication, and active substances and medicaments suitable therefor

(76) Inventors: Frank Becher, Koblenz (DE); Thomas Hille, Neuwied (DE); Frank Theobald, Bad Breisig (DE); Aharon Levy, Beit Hanan, IL (US)

Correspondence Address: D. PETER HOCHBERG CO. L.P.A., 1940 EAST 6TH STREET, CLEVELAND, OH, 44114, US

(21) Appl. No.: 10/542,515

(22) PCT Filed: Jan. 16, 2004
(86) PCT No.: PCT/EP04/00289

§ 371 Date: Jul. 18, 2005

(30) Foreign Application Priority Data
Jan. 17, 2003 (DE) 103 01 851.4

(51) Int. Cl. A61K 31/325

(52) U.S. Cl. 514/490

(57) ABSTRACT

(S)-N-ethyl-N-methyl-3-[1-(dimethylamino)ethyl]-phenyl carbamate (formula (1)) or at least one active ingredient according to formula (2) for the preventive protection of people from poisoning caused by cholinesterase inhibitors.

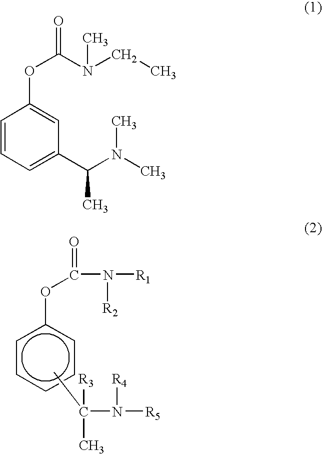

### CROSS-REFERENCE TO RELATED APPLICATIONS

This application is a National Stage application of International Application No. PCT/EP2004/000289, filed on Jan. 16, 2004, which claims priority of German application number 103 01 851.4, filed on Jan. 17, 2003.

### BACKGROUND OF THE INVENTION

1. Field of the Invention

The present invention relates to methods for the drug prophylaxis of poisoning caused by cholinesterase inhibitors, in particular those from the class of organophosphorus compounds. The present invention further relates to active ingredients and medicaments which are suitable as prophylactic agents/compositions for such poisoning, particularly medicaments containing active ingredients from the group of the phenyl carbamates. The invention further encompasses the use of the active ingredients for the prophylaxis of the poisoning.

2. Description of the Prior Art

Compounds with a cholinesterase-inhibiting effect are employed on the one hand as insecticides and fungicides in crop protection, and on the other hand some of these compounds are suitable for use as combat agents or combat gases in wars or in terrorist attacks. While in the latter case the toxic effect is intended, the poisoning of people caused by insecticides or fungicides is attributable to improper handling, especially inadequate safety measures during transport or during use.

The risk of being exposed to a poison gas attack has increased again recently because of terrorist activities. An additional factor is that some countries are producing or storing combat gases and are considering the use of such weapons to achieve their military aims. Those at risk are not only soldiers on combat operations but increasingly also the civil population and, in particular, the rescue services.

Nerve combat agents or nerve gases from the class of organic phosphoric acid esters and phosphonic acid esters are the most frequently used poison gases. The principal representatives of these combat agents are tabun (GA), sarin (GB), soman (GD), and VX.

Examples of representatives which may be mentioned of organophosphates having pesticidal or fungicidal activity and used in agriculture or horticulture are parathion (diethyl (4-nitrophenyl) thionophosphate), dimethoate (dimethyl S-methylcarbamoylmethyl dithiophosphate) and malathion.

The toxic effect of these combat agents, as well as that of the organophosphates and carbamates used as insecticides or fungicides, derives from inhibition of cholinesterase, resulting in an excessive accumulation of the neurotransmitter acetylcholine at the cholinergic receptors. The excessive activation of peripheral and central receptors causes severe paralytic symptoms, with death usually being caused by the respiratory paralysis which occurs. Further clinical symptoms are, for example, hypersalivation, apnoea and fits; these symptoms occur within the first few minutes after exposure to combat agents. If these symptoms are not treated adequately and immediately, they can cause death or permanent damage, comparable to the consequences of irreversible brain damage.

The antidote normally administered for the therapy of acute organophosphate poisoning is atropine in high parenteral doses in order to antagonize the effect of acetylcholine. This can take place with the aid of so-called autoinjectives which are intended to make it possible for the affected people in an emergency to self-administer the necessary atropine dose. However, such a treatment promises success only if it is undertaken, at the latest, within one minute after intake of the poison. In actual circumstances, this is possible in very rare cases because the time available in an emergency (e.g. in combat operations or terrorist attacks) is too short. This applies in particular to the extremely poisonous combat agents sarin (GB) and soman (GD). In addition, the atropine dose employed for the treatment must be carefully selected depending on the severity of the poisoning in order to avoid overdosage and atropine poisoning. In practice this is realistically hardly possible under the circumstances existing in the said operations.

It is possible in some cases to treat an organophosphate poisoning by administering oxime compounds (e.g. obidoxime, pralidoxime). However, oximes are effective only for certain alkyl phosphates (e.g. parathion), and the treatment must be undertaken as soon as possible after exposure to the poison. Oximes are effective for most organophosphates except for soman (GD), for example.

Only inadequate preventive means are available for the poisoning mentioned. One known example is oral administration of pyridostigmine, which was carried out in the second Gulf war as a measure to protect the soldiers from exposure to poison gas. However, this treatment has now been abandoned because pyridostigmine is suspected of causing serious side effects partly responsible for the Gulf War Syndrome. The compound pyridostigmine as such does not exhibit independent protective action. In the above-mentioned case, pyridostigmine was used only as pretreatment, not as a prophylactic agent. The intention of this pretreatment is to improve the actual treatment with the second active agent, the antidote atropine.

A further reason why pyridostigmine is no longer used is that to date there is no regular approval based on extensive clinical experiments proving the harmlessness of this medicament.

Furthermore, the currently available antidote therapies do not offer adequate protection from paroxysms caused by exposure to nerve gas, and from the long-term cerebral damage and cognitive disorders resulting therefrom.

DE 43 42 173 A1 proposed a combination of physostigmine and scopolamine as a prophylactic measure or for pre-treatment of organophosphate poisoning, the intention being to administer this combination by means of injection or skin plaster. However, it is a disadvantage that physostigmine is not approved as medicament. There are thus justified worries that physostigmine might, because of its chemical similarity to pyridostigmine, cause similar side effects as the latter.

### SUMMARY OF THE INVENTION

The object of the present invention is to indicate methods for prophylaxis of poisoning by cholinesterase inhibitors, and medicaments suitable for this purpose, it being intended to avoid or reduce the aforementioned disadvantages of known methods and medicaments.

### DETAILED DESCRIPTION OF THE INVENTION

In animal experiments (see the example) it has emerged, surprisingly, that this object is achieved by using (S)-N-ethyl-N-methyl-3-[1-(dimethylamino)ethyl]-phenyl carbamate (“compound (1)”) as prophylactic agent.

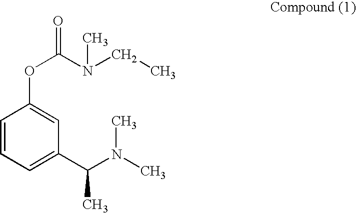

The solution according to the invention therefore encompasses methods for prophylactic treatment of poisoning caused by cholinesterase inhibitors, said methods being based on administration of (S)-N-ethyl-N-methyl-3-[1-(dimethylamino)ethyl]-phenyl carbamate or of a medicament containing said active ingredient. The invention further relates to the use of (S)-N-ethyl-N-methyl-3-[1-(dimethylamino)ethyl]-phenyl carbamate or of a medicament containing said active ingredient for the prophylaxis of poisoning caused by cholinesterase inhibitors. The invention furthermore encompasses medicaments containing the active ingredient (S)-N-ethyl-N-methyl-3-[1-(dimethylamino)ethyl]-phenyl carbamate in combination with at least one further pharmaceutical active ingredient.

Compound (1) is a carbamic acid ester which inhibits the enzyme cholinesterase by carbamylation. This inhibition is reversible with a half-life of a few minutes. Because of these properties, this active agent is employed for the therapy of Alzheimer's disease, the intention in this case being to compensate for the acetylcholine deficit caused by the destruction of cholinergic neurons. Compound (1) is approved as medicament for the treatment of Alzheimer's disease and is on the market; it causes an improvement in memory performance at least in some patients. The medicament is regarded as safe; no serious side effects are known. A further advantage is that (S)-N-ethyl-N-methyl-3-[1-(dimethylamino)ethyl]-phenyl carbamate is absorbed well from the gastrointestinal tract and easily crosses the blood-brain barrier.

The use of (S)-N-ethyl-N-methyl-3-[1-(dimethylamino)ethyl]-phenyl carbamate as a safe prophylactic for the prophylaxis of poisoning by cholinesterase inhibitors, especially organophosphate poisoning, has previously not been known. This specific particularly good suitability has surprisingly emerged in animal experiments. In these experiments it has emerged as a particular advantage that obviously a considerably lower dose is needed than in the normal therapy of Alzheimer's disease.

The medicaments of the invention for prophylactic treatment of organophosphate poisoning contain the active agent (S)-N-ethyl-N-methyl-3-[1-(dimethylamino)ethyl]-phenyl carbamate as a free base or in the form of a pharmaceutically acceptable acid addition salt. Particularly suitable salts are: salicylate, hydrogen tartrate, hydrobromide and hydrochloride. Particularly suitable is the hydrogen tartrate salt of the active agent (molecular formula C₁₄H₂₂N₂O₂·C₄H₆O₆), with the tartaric acid preferably being present in the configuration (2R, 3R).

The free active substance base or the acid addition salts thereof may be used as racemic mixtures; the (−)-enantiomer of (S)-N-ethyl-N-methyl-3-[1-(dimethylamino)ethyl]-phenyl carbamate and its acid addition salts are, however, preferred because of their greater selectivity. If the active ingredient is present in the form of an acid addition salt, the direction of rotation may be (+) or (−).

The free base (S)-N-ethyl-N-methyl-3-[1-(dimethylamino)ethyl]-phenyl carbamate can be obtained by amidation of □-m-hydroxyphenylethyl-dimethylamine with a corresponding carbamoyl halogenide. The separation of the racemates and the preparation of the acid addition salts may be accomplished according to known methods. The racemic mixture of the hydrochloride form of compound (1) is known from EP-A-0 193 926 (where it is designated as “RA₇HCl”). The (−)-enantiomer of compound (1) and its acid addition salts are described in DE 38 05 744 A1.

Suitable as active ingredients which according to the present invention may be utilized for the prophylactic treatment of poisoning caused by cholinesterase inhibitors are furthermore the compounds of the general structural formula (2):

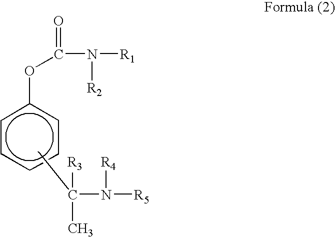

In this formula the residue R₁ is selected from the group consisting of hydrogen, straight-chain and branched lower alkyl residues (1 to 5 C atoms), cyclohexyl, allyl and benzyl; the residue R₂ is selected from the group consisting of hydrogen, methyl, ethyl and propyl; the residue R₃ is selected from the group consisting of hydrogen as well as straight-chain and branched lower alkyl residues (1 to 5 C atoms); the residues R₄ and R₅ are selected from the group of the straight-chain and branched lower alkyl residues (1 to 5 C atoms), with R₄ and R₅ being identical or different; the dialkylaminoalkyl group with the residues R₃, R₄ and R₅ can optionally be in ortho, meta or para position.

The active agent compounds according to formula (2) may be used as free bases or in the form of their pharmaceutically acceptable acid addition salts. The salts mentioned in connection with compound (1), particularly the hydrogen tartrate and the hydrochloride, are particularly suitable.

Compounds of the formula (2) and their production are disclosed in EP-A-0 193 926.

According to one embodiment of the invention, the compound (1) or one of the compounds according to formula (2), each, where appropriate, in the form of a pharmaceutically acceptable salt, is administered as the sole active agent for the prophylaxis of poisoning caused by cholinesterase inhibitors to persons who are at risk. This is a prophylactic measure which by itself is sufficient and does not necessitate any further therapeutic measures or active agents.

Another embodiment provides for the compound (1) or one of the compounds according to formula (2), each, where appropriate, in the form of a pharmaceutically acceptable salt, in combination (i.e. simultaneously or consecutively in time) with one or more additional active ingredient(s), to the person to be treated. This or these active ingredient(s) are selected from the group of the parasympatholytics, such as from the group of the tropane alkaloids, namely scopolamine. Further suitable active ingredients from this group are: atropine, butylscopolamine, benzatropine. These active ingredients may also be present in the form of their pharmaceutically acceptable salts.

The combination of compound (1) or of an active ingredient according to formula (2) with at least one active ingredient from the group of the parasympatholytics, in particular from the group of tropane alkaloids, may be employed because these active ingredients are competitive antagonists of released acetylcholine and thus reduce the unwanted effects caused by the cholinesterase-inhibitory effect of compound (1).

The present invention also encompasses medicaments with a content of compound (1) or of an active ingredient according to formula (2), where appropriate in the form of a pharmaceutically acceptable salt. This includes medicaments of the invention which contain such a phenyl carbamate active ingredient as the sole active ingredient component.

The medicament employed for prophylaxis may contain one or more additional active ingredient(s), as described above, said active ingredient(s) being selected from the group of the parasympatholytics.

The medicaments of the invention which contain compound (1) or at least one compound according to formula (2) can be produced using known adjuvants in various types of dosage forms. Pharmaceutical forms for enteral or for parenteral, particularly transdermal administration are employed in the prophylactic methods according to the invention. In the former case, the active ingredient(s) is/are present in an oral enteral dosage form (e.g. tablet, coated tablet, chewable tablet, suckable tablet, capsule, powder, suspension, solution) or in a rectal dosage form (e.g. suppository). Suitable formulation adjuvants are known to the skilled person.

Also suitable are parenteral oral administration forms such as suckable tablets, sublingual tablets, sheet-like adhesive systems applied to the oral mucosa, sheet-like systems that disintegrate on the tongue or in the oral cavity and administer the active agent by adhering to the oral mucosa.

Furthermore, devices for administering medicaments to mucosal tissue, as described in DE 0069030095 T2 and DE 0069032982 T2, are also suitable. These devices can be used like lollipops and substantially comprise a carrier device which is attached to a total mass.

Application by means of the “Methods and apparatus for using controlled heat to regulate transdermal delivery” described in US-A 2001037104 (Zhang Jie et al.) is also suitable.

The medicaments of the invention can, however, also be formulated as injection solutions and be present, for example, inside a disposable syringe. Depot pharmaceutical forms or therapeutic systems which make retarded and/or controlled release of active ingredient possible are particularly suitable.

It is particularly advantageous to add one or more antioxidants, such as those selected from the group consisting of tocopherol and its derivatives (especially esters, acetate), ascorbic acid and its derivatives (e.g. ascorbyl palmitate), butyl hydroxyanisol, butyl hydroxytoluene and propyl gallate, particularly α-tocopherol and ascorbyl palmitate. These substances are added in a concentration of 0.01 to approx. 1.0%-wt., or even 0.05 to 0.5%-wt., in each case relative to the entire pharmaceutical preparation.

The medicaments of the invention comprise 0.1 to 100 mg, particularly 0.5 to 20 mg, of compound (1) (or of an active agent according to formula (2)). In the case of oral single-dose forms, the active ingredient content is in the range from 0.1 to 10 mg, and in the case of depot pharmaceutical forms or therapeutic systems it is in the range from 1.0 to 100 mg. The content of the other active ingredient/active ingredients mentioned (such as tropane alkaloids) is in the range from 0.1 to 100 mg, particularly 0.5 to 50 mg. The percentage of active ingredient, relative to an individual pharmaceutical preparation, is in the range from 0.1 to 50%-wt., particularly in the range from 5 to 40%-wt.

The maximum daily dose (relative to the compound (1)) is approx. 2×6 mg per day (orally) or approx. 24 mg per day (transdermally).

According to one embodiment, the active agent (S)-N-ethyl-N-methyl-3-[1-(dimethylamino)ethyl]-phenyl carbamate (“compound (1)”), particularly in combination with at least one further active ingredient (especially scopolamine), is contained in a flat, film-like oral administration form. These administration forms, which are also known under the designation of “wafers”, are intended for application in the oral cavity. The medicament, with its active and inactive ingredients adopts a gel-like consistency upon access of saliva—or of another liquid—and thereby adheres to the oral mucosa, where the active agents are released and subsequently absorbed via the oral mucosa. During release, the wafer remains in the oral cavity, is virtually disintegrated and releases the active agent within a very short time.

In an alternative administration form, the wafer is provided with an adhesive layer, so that it adheres to the oral mucosa for a controlled, extended period of time.

Wafers substantially contain one or more polymers as base substances, as well as one or more active ingredient(s) which is/are dissolved or dispersed therein. Suitable polymers are, in particular, water-soluble polymers or polymers which are swellable or disintegratable in aqueous media. Particularly suitable are polymers selected from the following group: cellulose derivatives (such as hydroxypropyl methyl cellulose, carboxymethyl cellulose, hydroxypropyl cellulose and methyl cellulose); water-soluble polysaccharides of vegetable or microbial origin (such as pullulan, xanthan, alginates, dextranes, pectins, starch); polyvinyl alcohols, polyacrylates, polyvinyl pyrrolidone; proteins (such as gelatine or other gel-forming proteins).

Furthermore, the wafers may contain one or more additives, selected from the group of plasticizers, dyes and pigments, antioxidants, disintegration-promoting agents, wetting agents, absorption or permeation-promoting substances, pH regulators, filling agents, flavorings and aromatics, and sweeteners. Substances which are suitable for this purpose and which are pharmaceutically acceptable are known to the skilled person, the same applies to methods of production of such wafers (see e.g. DE-A-196 52 268; DE-A-100 32 456; WO-A-98 26 763). In the production of these wafers, a dispersion or solution of the components (polymer(s), active agent(s), additive(s)) is generally prepared first, and this is subsequently coated to a flat, inert carrier.

The thickness of these film-shaped administration forms is between 0.1 to 5 mm, or even between 0.5 to 1 mm.

An additional embodiment of the invention provides for the active substance or, particularly, an active substance combination as described above to be contained in a transdermal therapeutic system (TTS). Since compound (1) (as free base or as acid addition salt) as well as, for example, scopolamine have been shown to be able to penetrate the skin, these substances are suitable for the transdermal administration route.

Transdermal pharmaceutical forms are particularly advantageous for the prophylactic use of phenyl carbamate agents according to the present invention because they make precise control of active ingredient delivery possible over a prolonged period (up to 72 h), with the result that the dosage interval can be extended. In this manner it is possible to maintain a plasma concentration which is sufficient for the desired prophylactic effect without the occurrence of unfavourable peak plasma values or variations in the plasma concentration. For this reason, transdermal administration is also considerably more favourable in relation to the occurrence of side effects; in a few cases, the people treated by oral administration of compound (1) may experience nausea. The risk of overdosage is very substantially precluded with TTS; in addition, improved acceptance by the people to be treated can be expected.

The structure and the production of transdermal therapeutic systems (TTS) are in principle known to the skilled person. These systems comprise an active ingredient reservoir which may be either a membrane-enclosed, bag-like reservoir or a polymer-based reservoir (“matrix system”). The reservoir is normally connected to a support layer (e.g. plastic film such as PETP, PE; thickness 10-15 μm, for example) which serves as a backing layer during application and covers the active ingredient-containing reservoir toward the outside. The area of the active ingredient reservoir which faces the skin (delivery side) can optionally be covered before application with a detachable protective film (e.g. PE or PETP film, siliconized or fluorosiliconized; thickness, for example, 50-250 μm).

Polymers suitable for producing the active ingredient reservoir are, in particular, polymers from the following groups: polyacrylates, poly(meth)acrylates, polyacrylic acid, cellulose derivatives, in particular methyl- and ethyl celluloses, isobutylene, ethylene-vinyl acetate, natural and synthetic rubbers such as styrene-diene copolymers, styrene-butadiene block copolymers, isoprene block copolymers, acrylonitrile-butadiene rubber, butyl rubber or neoprene rubber, silicone pressure-sensitive adhesives and hot melt adhesives. Suitable pressure-sensitive adhesives are known to those skilled in the art (e.g. amine-resistant silicone pressure sensitive adhesives such as BIO-PSA® pressure sensitive adhesive, especially Q7-4302; Dow Corning). It is also possible and advantageous to use suitable mixtures of said polymers.

The term “hot melt adhesive” encompasses all adhesives which are liquefied not by solvents but by melting at elevated temperatures, for example in the range 60-200° C. Examples of suitable hot melt adhesives are mixtures of esters of hydrogenated colophony with cellulose derivatives.

The active ingredient reservoir of the TTS of the invention may further comprise various auxiliaries or additives, for example from the group of solubilizers, solvents, plasticizers, tackifiers, permeation-improving agents, pH regulators, antioxidants and preservatives. The polymer matrix of the active ingredient reservoir may be monolayer or multilayer; but having pressure-sensitive adhesive properties which enables a lasting contact of the active agent-releasing side of the reservoir to the skin. Alternatively, a separate active agent-free pressure-sensitive adhesive layer or a pressure-sensitive adhesive zone may be provided if the active ingredient reservoir has no or insufficient pressure-sensitive adhesive properties.

The typical structure of a TTS according to a preferred one embodiment encloses: a backing layer; (S)-N-ethyl-N-methyl-3-[1-(dimethylamino)ethyl]-phenyl carbamate (as base or as hydrogen tartrate) in an acrylate copolymer as active ingredient reservoir; silicone pressure-sensitive adhesive layer (BIO-PSA® Q7-4302); detachable protective layer.

As plasticizers, the substances from the following group are taken into consideration: eudermic tensides; polyoxyethylene fatty alcohol ethers, such as C₁₂-C₁₈ alcohol, especially polyoxyethylene(10) oleyl ether, especially Brij® 97 (Atlas Chemie); polyoxyethylene sorbitan fatty acid esters, preferably with C₁₂-C₁₈ fatty acids, especially preferably polyoxyethylene(20) sorbitan mono-oleate (e.g. Tween® 80 (Atlas Chemie); polyoxyethylene-(5-40) stearic acid esters (e.g. Myrj®; Atlas Chemie); polyoxyethylene glycol fatty alcohol ethers, e.g. polyethylene glycol(6-25)-cetyl ether, glycerol polyethylene ricinoleate; glycerol polyethylene glycol stearate (Cremophor®; BASF); polyoxyethylene glycol in a molecular weight range from 200 to 600 Dalton; Cetiol® HE (Henkel); lower alkyl esters of adipic acid, especially di-n-butyl adipate, diisopropyl adipate; glycerol polyethylene glycol ricinoleate (e.g. Cremophor EL, BASF®; triacetin-(1,2,3); fatty acids, fatty alcohols, in each case C₁₂-C₁₈.

As permeation-improving agents (enhancers), Azon (1-dodecyl azacycloheptan-2-one) or/and DEET (N,N-diethyl-m-toluamide) may be employed.

The entire constituent amount of plasticizers and permeation-enhancing substances may be up to approx. 50%-wt., relative to the active ingredient-containing polymer preparation (active ingredient reservoir). A content of less than 1%-wt. or complete absence of such additives may be employed.

The procedure for producing the TTS of the invention can be such that the compound (1) and/or an active ingredient of formula (2) as well as optionally further active ingredients is/are converted into a coarse, colloidal or molecular dispersion in a solution of matrix base polymers, and the mixture is coated onto a suitable substrate, for example a plastic film provided with a silicone layer. Examples of solvents which may be used are acetone, ethyl acetate or hexane, or solvent mixtures. After drying and evaporation of the solvent portions, the active ingredient-containing matrix layer is covered with another film which represents the later backing layer of the TTS. Individual TTSs are produced from such a laminate by punching out sheet-like structures in the desired geometric shape and size. Alternatively, production of the active ingredient-containing polymer matrix can start from a polymer melt, in which case the active ingredient-containing molten polymer mass is extruded in a thin layer onto a support in the form of a film. The active ingredient-containing layer is 10 μm to 2 mm, or even 50 μm to 0.5 mm. The skin contact area of a TTS may optionally be approx. 1 to 80 cm², or even approximately 2 to 20 cm².

If, as provided in the first embodiment, a TTS comprises the active ingredient (S)-N-ethyl-N-methyl-3-[1-(dimethylamino)ethyl]-phenyl carbamate (“compound (1)”) in combination with at least one other active ingredient (such as from the group of the tropane alkaloids, particularly scopolamine), it is possible to make use of the measure of constructing the TTS from a plurality of layers, surface areas, sections or compartments, in which case the individual layers, surface areas, sections or compartments differ through the nature or/and concentration of the active ingredient present.

A TTS according to one embodiment may, for example, contain two compartments which serve as a reservoir for the phenyl carbamate agent (compound (1) or/and compound according to formula (2)) and for scopolamine, respectively. These two reservoirs are connected with a shared backing layer and protective layer. The relative surface area dimensions of the two compartments (and/or the relative amounts and concentrations of the active agents) can be adapted correspondingly in order to adjust the permeation rate of each one of the active agents. Thus, the first compartment (with a content of compound (1), e.g. 60 mg) may, for example, have an area of 25 cm²; in this case the second compartment (with scopolamine; e.g. 4 mg) has an area of 7.5 cm².

As mentioned above, the present invention also comprises TTSs which are configured as bag-like systems. In this case the active ingredient(s) is/are contained in a liquid or semi-liquid (e.g. gel-like or viscous) composition which is enclosed in a bag-shaped container. The release of active ingredient takes place via an adhesively coated membrane of the container, which membrane comes into contact with the skin of the person to be treated. Suitable materials and methods for producing such systems are in principle known to those skilled in the art.

Transdermal systems which are particularly advantageously suitable for the prophylactic administration of compound (1) or active ingredients according to formula (2), possibly in combination with at least one further active ingredient, are disclosed in WO-A-99 34782. In the context of the present invention, express reference is therefore made to the pharmaceutical compositions and components described in WO-A-99 34782.

In accordance with WO-A-99 34782 the active ingredient(s) is/are dissolved in one or more matrix polymers, such as hydrophile polymers. These polymers are selected from the group of the polyacrylates and polymethacrylates; their mean molecular weight being in the range from approx. 50,000 to approx. 300,000 Dalton. These are, in particular, polymers with film-forming properties.

Substances which may come into consideration are acrylate copolymers, e.g. copolymers of butyl acrylate, ethyl hexyl acrylate and vinyl acetate. The use of cross-linked polymers of the type mentioned is also of particular advantage. Examples of particularly suitable polymers to be mentioned are Durotak 87-2353, Durotak 387-2051 and Durotak 387-2052 (available from: National Starch and Chemical Company). The portion of these polymers may be up to 90%-wt, preferably up to 70%-wt., in each case relative to the total weight of the active ingredient-containing preparation.

Substances suitable as hydrophile polymers are, in particular, polyacrylamide and its copolymers, polyvinyl pyrrolidones, polyvinyl alcohol and derivatives thereof, vinyl acetate-vinyl alcohol copolymers, ethyl cellulose and other cellulose and starch derivatives. Hydrophile polyacrylates may be employed; the polyacrylate may be substituted (e.g. a methacrylate), likewise some or all acid groups may be esterified, e.g. with alkyl(C₁ to C₁₀) groups, especially with methyl or ethyl groups. Examples for commercially available polymers of this type are: Plastoid® B (by Röhm, Darmstadt); Eudragit® RS 100 and RL 100 (Röhm); Eudragit® E 100 (Röhm).

Additionally, hydrophobic polymers may be contained, especially one or more synthetic resins, possibly in combination with modified substances such as colophonic acids, glyceryl esters and phthalate esters of colophonic acids.

TTSs with an active ingredient reservoir which has the following composition: 20 to 40%-wt. of (S)-N-ethyl-N-methyl-3-[1-(dimethylamino)ethyl]-phenyl carbamate (as free base or as salt, particularly as hydrogen tartrate); 10 to 30%-wt. of polymethacrylate; 40 to 60%-wt. of an acrylate copolymer; 0.05 to 0.3%-wt. of □-tocopherol (total: 100%-wt.) may also be employed.

The medicaments of the present invention containing compound (1) or/and at least one active ingredient according to formula (2) are advantageously suitable for the prophylactic treatment of poisoning caused by cholinesterase inhibitors, in particular toxic substances of the type mentioned at the outset. Cholinesterase inhibitors means in general compounds capable of chemical modification of the active centre of the enzyme, in particular through reaction with hydroxyl groups in the active centre. These are primarily organophosphorus compounds such as organic phosphoric acid esters and organic phosphonic acidesters and derivatives thereof in each case. In addition, also suitable in connection with the present invention are cholinesterase inhibitors from other substance classes, e.g. carbamates, in particular those used as crop protection agents (e.g. carbaryl=1-naphthyl N-methyl carbamate).

The prophylactic agents or compositions of the invention can be employed in agriculture or horticulture in order to protect the workers who must handle the organophosphorus insecticides or fungicides, or who may come into contact therewith, from possible poisoning. These prophylactic medicaments are likewise suitable for protecting people employed for weapon decommissioning work or for decontamination work. The invention further includes the use of (S)-N-ethyl-N-methyl-3-[1-(dimethylamino)ethyl]-phenyl carbamate or of a compound according to formula (2) for the prophylactic treatment of soldiers, police officers and civilians for protection from the combat agents or nerve gases mentioned.

The protective effect reduces the toxicity and improves the chances of survival after exposure to toxic substances. It also increases the prospects of successful post-exposure therapy with an atropine-oxime combination.

The present invention also relates to methods for the prophylactic treatment or pretreatment of people in order to protect them from poisoning caused by exposure to organophosphorus cholinesterase inhibitors. These methods are distinguished by comprising at least one step in which a medicament containing (S)-N-ethyl-N-methyl-3-[1-(dimethylamino)ethyl]-phenyl carbamate or/and an active ingredient according to formula (2) (in each case possibly in the form of a pharmaceutically acceptable salt) is administered to a person. The active ingredient-containing medicaments as described above are used in this connection.

Prophylactic administration of medicaments containing at least one of the above-mentioned phenyl carbamates is carried out at least one day, and in some circumstances only at least 2 hours, before the expected exposure to toxic substances, where a predictable event is involved (e.g. handling of insecticides, decontamination work, starting a combat operation). The protective effect can be maintained by administration of a plurality of consecutive single doses, such as by administration of depot pharmaceutical forms or therapeutic systems (particularly TTS), over a period of several (1 to 7) days up to some weeks.

In an alternative embodiment, at least one medicament which comprises (S)-N-ethyl-N-methyl-3-[1-(dimethylamino)ethyl]-phenyl carbamate or a pharmaceutically acceptable salt of that compound as sole active ingredient is administered in the prophylactic method.

A further alternative embodiment of the prophylactic method provides for additional administration of one or more other active ingredients from the group of the parasympatholytics to the person to be treated, such as active ingredient(s) from the group of the tropane alkaloids, particularly scopolamine. This combined administration can take place either by administration of a medicament which comprises said active ingredients in combination, or by simultaneous or successive administration of individual medicaments each of which comprises only one active ingredient of the combination of active ingredients. For example, prophylactic treatment of a person is possible by application of a TTS containing (S)-N-ethyl-N-methyl-3-[1-(dimethylamino)ethyl]-phenyl carbamate to the skin (e.g. the upper arm) and during this application time, oral administration of a second medicament, which preferably comprises scopolamine, to this person. An alternative possibility is treatment by application of a TTS which comprises a combination of at least two active ingredients (e.g. (S)-N-ethyl-N-methyl-3-[1-(dimethylamino)ethyl]-phenyl carbamate and scopolamine) to the skin. There is also provision further for combination of transdermal or oral administration of (S)-N-ethyl-N-methyl-3-[1-(dimethylamino)ethyl]-phenyl carbamate with parenteral administration of at least one further active ingredient, such as from the group of parasympatholytics.

In the case of transdermal administration of the active ingredients (as described above), the protective effect occurs after approx. 4 h at the earliest. This delay can in certain circumstances be critical, e.g. in the case of a terrorist attack, where immediate intervention by soldiers or police is required. To achieve an earlier onset of protective action, the present invention includes a method for prophylactic treatment which combines transdermal administration and oral administration of the above-mentioned active ingredients. In a first step, (S)-N-ethyl-N-methyl-3-[1-(dimethylamino)ethyl]-phenyl carbamate (or a combination of that active substance and scopolamine) is administered via the oral route to a person for whom a quick onset of the protective effect is required. In this manner it is possible to achieve therapeutic plasma levels within a short period of time (within approx. ½ h) which offer protection from combat agents. This enables the treated person to access a contaminated area immediately after receiving the emergency deployment order.

In a second step, a transdermal system (as described above) is administered to the same person in order to achieve a lasting protective effect (e.g. up to 24 hours). Application of the TTS may take place at the same time as administration of the oral medicament; it may, however, also be carried out after a delay in time, such as within 12 hours after oral administration. This second step may be repeated in certain time intervals (e.g. 6 to 24 hours) to prolong protective action. According to this method, oral administration is necessary only in order to achieve an early onset of protective action; maintaining the protective effect is made possible by administering one or more transdermal therapeutic systems. This method is particularly uncomplicated and safe in handling; it enables a quick build-up of a prophylactic protective effect in the persons to be protected without subjecting those persons to unacceptable stress caused by side effects.

Oral administration forms for use in the methods described above are tablets and, particularly, “wafers” (film-like or wafer-like administration forms; as described above). According to one embodiment of the process, a wafer containing compound (1) and scopolamine is applied into the oral cavity of a person. The active substances are released from the wafer and absorbed via the oral mucosa. A therapeutic, prophylactic plasma level that ensures a protective effect is quickly achieved (e.g. within 30 min).

The prophylactic agents/compositions and methods of the invention are advantageously suitable for the pretreatment of people at risk of exposure to toxic substances.

### EXAMPLE

To show the prophylactic property of the combination of (S)-N-ethyl-N-methyl-3-[1-(dimethylamino)ethyl]-phenyl carbamate (compound (1)) and scopolamine against nerve poisons, the protective action was examined in a study on piglets as an animal model. The plasma levels of the two active ingredients were adjusted in the animals so as to be within a range corresponding to that which is to be used in humans.

In a preliminary test, 6 mg (S)-N-ethyl-N-methyl-3-[1-(dimethylamino)ethyl]-phenyl carbamate hydrogen tartrate was administered to the piglets (12 kg each) orally in the form of capsules. This caused a 20-40% inhibition of the enzyme cholinesterase in the blood two hours after administration. This range corresponds to the inhibition which is aimed at and is to be achieved in humans. In this context, it has to be taken into consideration that in humans a lower dose is required on account of the better absorption of the active agent. Based on the blood cholinesterase inhibition measured in humans it can be assumed that a treatment with 3 mg BID (i.e. twice daily) is sufficient to ensure the necessary protective effect. This dose is thus considerably smaller than the dose employed for treatment of Alzheimer patients (12 mg per day; Culter N R et al.: Dose-dependent CSF acetylcholinesterase inhibition by SDZ ENA 713 in Alzheimer's disease. Acta Neurol. Scand., 1998, 97, 244-250).

In a further preliminary experiment, it has emerged that intravenous infusion of scopolamine at a rate of 0.8 mg/kg/h leads to an equilibrium concentration of approx. 150 pg/ml in the piglets' blood; here, too, this value corresponds to the concentration which according to expectation is to be achieved in humans.

In the experiments on protective effect, the piglets were subjected to a dose of the nerve combat agent sarin which corresponded to double the LD₅₀ dose (40 μg/kg); this was done by means of an intravenous cannula in an ear of the piglets. Untreated piglets (control) died within 4 to 6 minutes after exposure to the combat agent. Treated piglets (each approx. 12 kg) were administered a 6 mg capsule (S)-N-ethyl-N-methyl-3-[1-(dimethylamino)ethyl]-phenyl carbamate hydrogen tartrate; in addition, the piglets were infused with scopolamine (0.8 mg/kg/h) 2 hours prior to exposure. Before treatment with active agent and immediately before exposure, blood samples were taken from the Vena subclavia to determine scopolamine and to assess cholinesterase inhibition.

As can be seen from the following table, all five piglets survived. Furthermore, the average recovery time, during which the piglets were standing firmly on their feet, was extraordinarily short (17 min) despite the relatively high dose of nerve combat agent.

TABLE
Protection of pigs from sarin (2 × LD₅₀) by prophylactic treatment
with (S)-N-ethyl-N-methyl-3-[1-(dimethylamino)ethyl]-phenyl carbamate
hydrogen tartrate (orally) and scopolamine (infusion)

| Surviving | Average recovery | Average ChE | Average scopol. conc. |
|---|---|---|---|
| animals | time [min.] | inhibtion [%] | [pg/ml] |
| 5/5 | 17 | 28 | 240 |

ChE = Cholinesterase

What has been described above are preferred aspects of the present invention. It is of course not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, combinations, modifications, and variations that fall within the spirit and scope of the appended claims.

1.-24. (canceled)

25. A medicament for the prophylaxis of poisoning by cholinesterase inhibitors, containing at least one active ingredient according to the following formula (2) 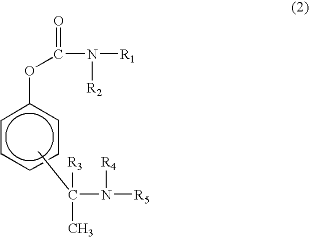
wherein,
R₁ is selected from the group consisting of hydrogen, straight-chain and branched lower alkyl residues (1 to 5 C atoms), cyclohexyl, allyl and benzyl;
R₂ is selected from the group consisting of hydrogen, methyl, ethyl and propyl;
R₃ is selected from the group consisting of hydrogen and straight-chain and branched lower alkyl residues (1 to 5 C atoms);
wherein said residues R₄ and R₅ are selected from the group consisting of straight-chain and branched lower alkyl residues (1 to 5 C atoms), said R₄ and R₅ being identical or different; and wherein the dialkyl aminoalkyl group with the residues R₃, R₄ and R₅ being in a position selected from the group consisting of ortho, meta and para position;
said active ingredient being present in a form selected from the group consisting of a free base and a pharmaceutically acceptable salt;
and wherein said medicament comprises at least one additional active ingredient selected from the group consisting of parasympatholytics.

26. The medicament according to claim 25 wherein said active ingredient comprises (S)-N-ethyl-N-methyl-3-[1-(dimethylamino)ethyl]-phenyl carbamate having the following formula (1)

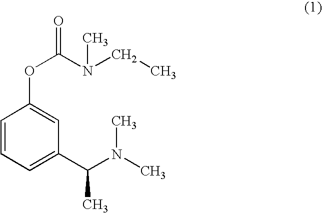

27. The medicament according to claim 25, wherein said at least one active ingredient according to formula (2) is present as an acid addition salt.

28. The medicament according to claim 27, wherein said acid addition salt is selected from the group consisting of hydrochloride and hydrogen tartrate.

29. The medicament according to claim 25, wherein said medicament is selected from the group consisting of orally, rectally and transdermally administrable medicaments.

30. The medicament according to claim 25, wherein said medicament is present in a form selected from the group consisting of a transdermal therapeutic system and a film-like oral dosage form.

31. The medicament according to claim 25, wherein said medicament comprises said at least one active ingredient in an amount of 0.1 to 100 mg.

32. The medicament according to claim 25, wherein said at least one additional active ingredient is selected from the group consisting of tropane alkaloids.

33. The medicament according to claim 32, wherein said at least one additional active ingredient is scopolamine.

34. A method for the prophylactic treatment of people against poisoning caused by exposure to cholinesterase inhibitors selected from the group consisting of organophosphorus compounds and carbamates, wherein said method comprises at least one step of administering a medicament to a person who is to be protected, said medicament comprising at least one active ingredient according to the following formula (2) 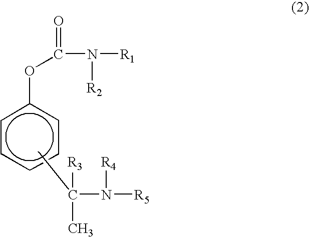
in a form selected from the group consisting of a free base and a pharmaceutically acceptable salt.

35. The method according to claim 34 wherein said active ingredient comprises (S)-N-ethyl-N-methyl-3-[1-(dimethylamino)ethyl]-phenyl carbamate having the following formula (1)

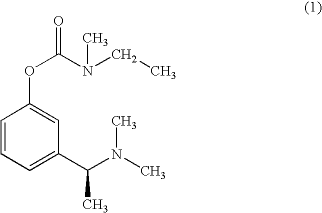

36. The method according to claim 35, wherein said medicament comprises said active ingredient (S)-N-ethyl-N-methyl-3-[1-(dimethylamino)ethyl]-phenyl carbamate (1) as the sole active ingredient.

37. The method according to claim 34, wherein said at least one active ingredient according to formula (2) is the sole active ingredient for prophylaxis.

38. The method according to claim 34, further comprising the step of administering to the person to be treated at least one additional active ingredient selected from the group consisting of parasympatholytics.

39. The method according to claim 34, further comprising the step of administering to the person to be treated at least one additional active ingredient by a combination preparation containing at least one active ingredient according to formula (2) and at least one active ingredient selected from the group consisting of parasympatholytics.

40. The method according to claim 39, wherein the combination preparation contains the active ingredient (S)-N-ethyl-N-methyl-3-[1-(dimethylamino)ethyl]-phenyl carbamate (1).

41. The method according to claim 34, wherein at least one of said at least one active ingredient is administered in a route selected from the group consisting of orally and transdermally.

42. The method according to claim 34, wherein said poisoning is caused by the uptake of at least one substance selected from the group consisting of organic phosphoric acid esters, organic phosphonic acid esters, derivatives of organic phosphoric acid esters and derivatives of organic phosphonic acid esters.

43. The method according to claim 34, wherein said poisoning is caused by crop protection agents.

44. The method according to claim 34, wherein said poisoning is caused by a poison selected from the group consisting of combat agents and nerve gases.

45. The method according to claim 41, comprising the steps of:
introducing a film-like oral administration form (wafer) into the oral cavity of a person, or administering a tablet, pill, capsule or coated tablet to the person; and
applying a transdermal therapeutic system to the skin of the person,
wherein said medicaments comprise said at least one active ingredient according to formula (2), and wherein said at least one active ingredient is present in the form selected from the group consisting of a free base and a pharmaceutically acceptable salt.

46. The method according to claim 45 wherein said active ingredient comprises (S)-N-ethyl-N-methyl-3-[1-(dimethylamino)ethyl]-phenyl carbamate of formula (1).

47. The method according to claim 45, wherein at least one of said medicaments administered to the person further comprise at least one additional active ingredient selected from the group of parasympatholytics.

48. The medicament according to claim 47, wherein said at least one additional active ingredient is selected from the group consisting of tropane alkaloids.

49. The medicament according to claim 48, wherein said at least one additional active ingredient is scopolamine.

50. The method according to claim 47, further comprising the step of administering to the person to be treated at least one additional active ingredient by a combination preparation containing at least one active ingredient according to formula (2) and at least one active ingredient selected from the group consisting of parasympatholytics.
